(19) United States
(12) Patent Application Publication
(10) Pub. No.: US 2010/0121057 A1
(43) Pub. Date: May 13, 2010

(54) Process for the Preparation of Piperazine Benzothiazoles

(75) Inventors: Cesare Schiavo, Killingworth (GB); Marco Poma, Rome (IT); WIlliam Nadler, Rome (IT)

Correspondence Address: SALIWANCHIK LLOYD & SALIWANCHIK;A PROFESSIONAL ASSOCIATION, PO Box 142950, GAINESVILLE, FL, 32614, US

(73) Assignee: Merck Serono SA, Coinsins Vaud (CH)

(21) Appl. No.: 12/595,839

(22) PCT Filed: Apr. 3, 2008
(86) PCT No.: PCT/EP2008/054055

§ 371 Date: Oct. 14, 2009

Related U.S. Application Data
(60) Provisional application No. 60/912,198, filed on Apr. 17, 2007.

(30) Foreign Application Priority Data
Apr. 17, 2007 (EP) 07106346.5

(51) Int. Cl. C07D 417/14; C07D 241/04

(52) U.S. Cl. 544/295; 544/391

(57) ABSTRACT

The present invention discloses a process for the preparation of compounds of formula (I)

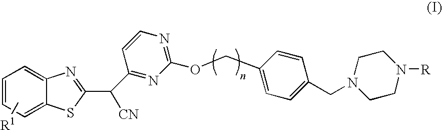

where the groups and symbols are as defined in the description, said process comprising a) reacting a benzothiazol-2-ylacetonitrile bearing group R₁ with an activated pyrimidine, in a reaction medium; then treating the obtained derivative in said reaction medium with a weak base anion exchange resin; then, after removing said resin and isolating the reaction product, reacting it with a substituted piperazine-benzyl-alkyloxy. The final product can optionally be salified. The invention also relates to the preparation of N-acyl-substituted piperazine-benzyl-alkyloxy, comprising treating a bromide-alkyl-phenyl-4-ester wherein the ester group is selected from COOMe or COOEt with DIBAL to obtain (4-bromomethyl-phenyl)-methanol and reacting the latter with 1-piperazin-1-yl-acyl. The processes here disclosed present a number of advantages, for example, higher yield and purity of final product. Moreover, in case of piperazine group bearing hydrogen or an acyl group in position 4, the preparation of the intermediate substituted piperazine-benzyl-alkyloxy (V) need no protection/deprotection steps.

The present invention relates to a process for the preparation of piperazine benzothiazoles.

Piperazine benzothiazole derivatives are disclosed in WO 03/091249 as medicaments, in particular for treatment and/or prophylaxis of cerebral ischemic disorders or CNS disorders.

The compounds are disclosed by the following general formula

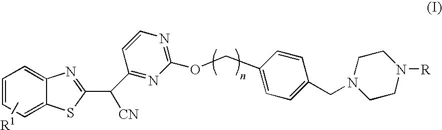

wherein
R is selected from the group consisting of hydrogen, C₁-C₆ alkyl, C₁-C₆ alkyl aryl, aryl-C₁-C₆ alkyl, heteroaryl, C₁-C₆ alkyl heteroaryl, heteroaryl-C₁-C₆ alkyl, C₂-C₆ alkenyl, C₂-C₆ alkenyl aryl, aryl-C₂-C₆ alkenyl, C₂-C₆ alkenyl heteroaryl, heteroaryl-C₂-C₆ alkenyl, C₂-C₆ alkynyl, C₂-C₆ alkynyl aryl, aryl-C₂-C₆ alkynyl, C₂-C₆ alkynyl heteroaryl, heteroaryl C₂-C₆ alkynyl, C₃-C₈ cycloalkyl, heterocycloalkyl, C₁-C₆ alkyl cycloalkyl, cycloalkyl-C₁-C₆ alkyl,C₁-C₆ alkyl heterocycloalkyl, heterocycloalkyl-C₁-C₆ alkyl, C₁-C₆ alkyl carboxy, carboxyC₁-C₆ alkyl, acyl, C₁-C₆ alkyl acyl, oxy-acyl, C₁-C₆ alkyloxyacyl, alkoxy, alkoxycarbonyl, alkylcarboxycarbonyl, aminocarbonyl, C₁-C₆ alkyl aminocarbonyl, aminoacyl, ureido, oxysulfonyl, C₁-C₆ alkyl oxysulfonyl, C₁-C₆ alkyl sulfonyl, C₁-C₆ alkyl sulfinyl, —C₆ alkyl sulfanyl, amino sulfonyl, C₁-C₆alkyl-aminosulfonyl;
R¹ is selected from the group consisting of hydrogen, halogen, cyano, nitro, amino, C₁-C₆ alkyl, heteroaryl, C₂-C₆ alkenyl, C₂-C₆ alkenyl aryl, C₂-C₆ alkenyl heteroaryl, C₂-C₆ alkynyl, C₁-C₆ alkyl aryl, aryl or heteroaryl, C₁-C₆ alkyl heteroaryl, —C(O)—OR², —C(O)—R², —NR²R²′, —S(O₂)—R², with
R² and R²′ being independently selected from the group consisting of hydrogen, C₁-C₆ alkyl, C₂-C₆ alkenyl, C₂-C₆ alkynyl, aryl, heteroaryl, C₁-C₆-alkyl aryl,aryl-C₁-C₆-alkyl C₁-C₆-alkyl heteroaryl, heteroaryl-C₁-C₆-alkyl;
n is an integer from 0 to 3.

The above formula (I) also includes tautomers, geometrical isomers, optically active forms as enantiomers, diastereoisomers and racemates, as well as pharmaceutically acceptable salts.

The above mentioned reference also discloses compounds of formula (II), which are a tautomeric form of compounds of formula (I).

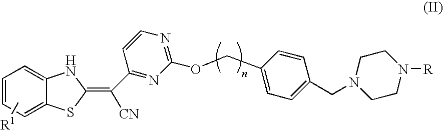

The above mentioned reference also discloses a process for the preparation of the compounds of formula (I) or (II).

The process therein disclosed comprises basically two steps. In a first step, a benzothiazoleacetonitrile, bearing group R₁ (III) is reacted with an activated pyrimidine (VI), such as a dihalogenopyrimidine, in order to provide a halogeno-pyrimidino-benzothiazole derivative (IV). In the second step, the derivative (IV) is reacted with a suitable substituted piperazine-benzyl-alkyloxy (V), bearing the piperazine group.

The synthesis is illustrated in the scheme below.

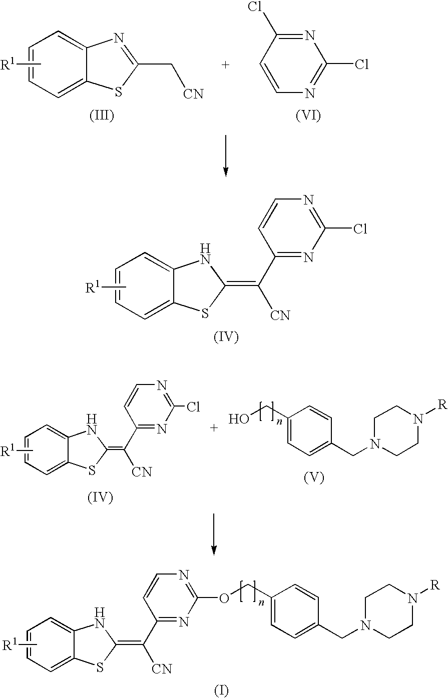

A critical step is represented by the preparation of substituted piperazine-benzyl-alkyloxy (V), when not commercially available.

The above reference teaches the preparation of substituted piperazine-benzyl-alkyloxy (V) through a synthetic approach shown in the scheme below, in the preferred embodiment of a benzyl alcohol, being intended that for compounds (V) where n is 2 or 3, the corresponding methyl p-ethyl-or p-propylbenzoate can be used.

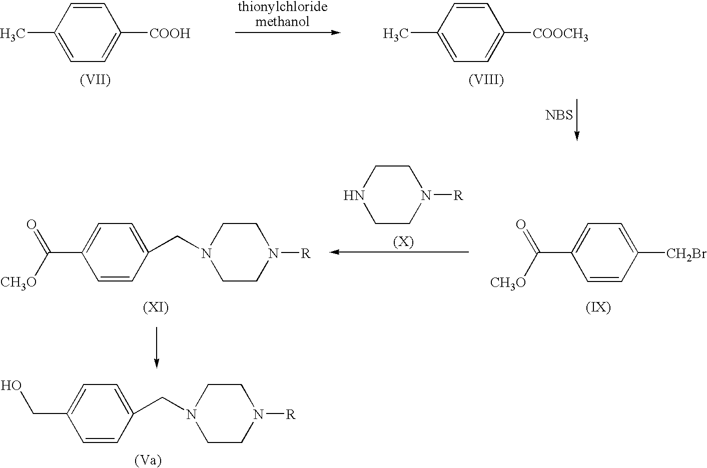

The synthetic methods above illustrated allow the preparation of the compounds of formula (I) in reasonable good yields.

However, for scaling up the process to industrial level, there is still room for further improvements.

For example, in the prior process, the reaction of compound (III) with compound (VI) leads to a mixture of regioisomers, which is isolated as solid product before subsequent step. This implies multiple filtrations and slurries.

Moreover, in case of piperazine group bearing hydrogen or an acyl group in position 4, particular precautions must be taken in the preparation of the intermediate substituted piperazine-benzyl-alkyloxy (V). In fact, as taught in the above mentioned reference, protection/deprotection steps for nitrogen atom in position 4 of the piperazine ring must be carried out.

The present invention aims to provide a process which overcomes the drawbacks of the prior art.

### SUMMARY OF THE INVENTION

It has now been found a process for the preparation of the above described compounds of formula (I) which avoids the isolation of the regioisomer mixture, improves workability of reactions involved, provides the desired compounds of formula (I) with better yields and is suitable for large scale production. Moreover, a process has been found for the preparation of substituted piperazine-benzyl-alkyloxy (V) which avoids protection/deprotection steps for piperazine rings bearing hydrogen or acyl group in nitrogen at position 4.

In one aspect the present invention provides a process for the preparation of compounds of formula (I), above described comprising the following steps:
a) reacting a benzothiazol-2-yl-acetonitrile bearing group R₁ (III) with an activated pyrimidine (VI), such as a dihalogenopyrimidine to obtain a halogeno-pyrimidino-benzothiazole derivative (IV) in a reaction medium such as acetonitrile (CAN) or N-methylpyrrolidone (NMP);
b) treating the derivative (IV) obtained in step a) in said reaction medium with a weak base anion exchange resin such as Duolite A7;
c) removing said resin from said reaction medium;
d) isolating compound (IV);
e) reacting compound (IV) with a substituted piperazine-benzyl-alkyloxy (V), to give a compound of formula (I);
f) optionally salifying compound (I).

In another aspect the present invention provides a process for the preparation of a substituted piperazine-benzyl-alkyloxy of formula (V) wherein R is an acyl group, said process comprising:
a) treating a bromide-alkyl-phenyl-4-ester wherein the ester group is selected from COOMe or COOEt with DIBAL to obtain (4-bromomethyl-phenyl)-methanol;
b) reacting the (4-bromomethyl-phenyl)-methanol obtained in step a) with 1-piperazin-1-yl-acyl.

In a further aspect, the present invention provides a process for the preparation of compounds of formula (I), wherein R is acyl, comprising the following steps:
a) treating a bromide-alkyl-phenyl-4-ester wherein the ester group is selected from COOMe or COOEt with DIBAL to obtain (4-bromomethyl-phenyl)-methanol;
b) reacting the (4-bromomethyl-phenyl)-methanol obtained in step a) with 1-piperazin-1-yl-acyl to give 1-[4-(4-ydroxymethyl-benzyl)-piperazin-1-yl]-acyl (V);
c) reacting said 1-[4-(4-hydroxymethyl-benzyl)-piperazin-1-yl]-acyl (V) obtained in step b) with a halogeno-pyrimidino-benzothiazole derivative (IV) to give a compound of formula (I);
d) optionally removing group R and/or optionally salifying compound (I).

These and other aspects of the present invention will be now described in detail and also by means of examples.

### DETAILED DESCRIPTION OF THE INVENTION

As to the definitions of the groups of formula (I), in particular R, R¹, R² and R^{2′}, specific reference is made to the above mentioned WO 03/091249.

In a first preferred group of compounds of formula (I), R represents acyl, more preferably acetyl and n is 1.

In a second preferred group of compounds of formula (I) R represents acyl, more preferably acetyl, n is 1 and R¹ is hydrogen.

The most preferred compound of formula (I) is the one wherein R is acetyl, n is 1 and R¹ is hydrogen, namely [2-({4-[(4-acetylpiperazin-1-yl)methyl]benzyl}oxy)-pyrimidin-4-yl](1,3-benzothiazol-2-yl)acetonitrile.

Salts of the compounds of formula (I) are preferably pharmaceutically acceptable salts, such as disclosed for example in Wermuth, C. G. and Stahl, P. H. (eds.) Handbook of Pharmaceutical Salts, Properties; Selection and Use; Verlag Helvetica Chimica Acta, Zürich, 2002. Preferred salts are dimethanesulfonate, mesylate and trifluoracetate.

In the present invention, the groups COOMe and COOEt have the conventional meanings of the art of methyl (COOCH₃) and ethyl (COOCH₂CH₃) esters.

In the process according to the present invention, acetonitrile is a suitable reaction medium for step a). The reaction is carried out at a temperature compatible with reactants, for example room temperature and for a suitable reaction time, for example about 24 hours. The reaction is carried out at the presence of a hydride, such as NaH. After completion, the reaction can be quenched with an aqueous solution, for example 20% aqueous NaCl. The organic impurities are extracted from the aqueous environment, for example with a hydrocarbon solvent, for example heptane, optionally followed by a further extraction, for example with isopropyl alcohol.

The reaction medium is then re-established at about the same starting conditions, for example removing water by azeo-drying.

Reaction mixture is then treated with a weak base anion exchange resin. Duolite A 7 is a preferred resin. A person skilled in the art is comfortably aware of the exchange resin technology, therefore the selection of the appropriate exchange resin suitable to the purposes of the present invention is within the normal practice of the person of ordinary skill in the art. For a general description on exchange resin technology, reference can be made to CRC Handbooks, for example Robert E. Smith “Ion Chromatography Applications”, Joseph Sherma (Ed.) “Handbook of Chromatography”, U.S. Pat. No. 4,170,628.

This treatment with anion exchange resin is carried out in the presence of an organic or inorganic base at a temperature compatible with reactants till completion of reaction. Removal of the resin can be carried out according to the usual knowledge, for example by filtration. Desired material can be isolated after lowering the pH of the solution (e.g. using aqueous HCl) obtained after resin removal by filtration of the precipitated solid.

Also compound (IV) isolation can be carried out as disclosed in the above reference or by any other well-known procedure known by a person skilled in the art.

Step e) can be carried out as disclosed in the above mentioned reference, as well as optional salification is done as known by a person skilled in the art.

It has been found that step e) provides higher yield of the final product and also with a higher purity is the reaction medium is made of N-methylpyrrolidone (NMP).

A further object of the present invention is a process for the preparation of substituted piperazine-benzyl-alkyloxy of formula (V), as described above.

According to the present invention, the 4-bromomethyl-benzoic acid methyl ester is treated with DIBAL to give the corresponding (4-Bromomethyl-phenyl)-methanol, which is then reacted with the desired 4-acylpiperazine, as better illustrated in the following scheme for the preferred embodiment of the benzyl alcohol and of the preferred embodiment of 4-acetyl-piperazine.

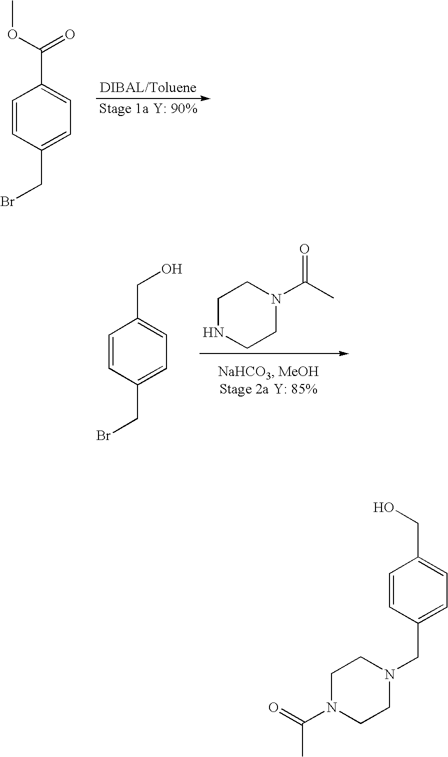

It is intended that the scheme below is sufficient to the skilled person to carry out this aspect of the invention in its whole breadth, only resorting to the general knowledge in this field of organic chemistry. This is evident looking at formula (V) above, where for values of n different from 1, shown in the example, commercially available starting material are suitably selected or can be prepared with method of normal practice in organic chemistry. The same applies for acyl groups on the piperazine nitrogen.

The advantages over the processes of the prior art are evident in terms of easiness of the reaction and increase of yield.

The following example further illustrates the present invention.

### Example 1

### a. Preparation of intermediate (IV) Benzothiazol-2-yl-(2-chloro-pyrimidin-4-yl)-acetonitrile

In a vessel, 2,4-dichloropyrimidine was added portion wise to 10 volumes of acetonitrile, stirring at 25° C. to obtain a clear solution (solution B).

(1,3-benzothiazol-2-yl)acetonitrile was added portion wise under stirring to solution B in about 15 minutes, stirring at about 25° C.

In a glass-lined vessel (A), NaH under N₂ was charged to 6.5 vols of acetonitrile at 20° C., under stirring. Then, solution B was charged to vessel A under stirring keeping temperature below 25° C. under stirring over 90-120 minutes.

The slurry was stirred at about 25° C. for 18-20 hours.

The reaction was monitored by HPLC.

H₂O (10 vols) was charged portion wise to vessel A under stirring keeping temperature below 30° C.

Heptanes (3×3.5 vols) were added under stirring at 25° C. Heptane layer and supernatant were removed each time leaving the aqueous layer.

15% w/w NaCl aqueous solution was added and stirred for 15 minutes. Water phase was transferred from vessel A into another vessel, wherein 3.5 volumes acetonitrile were charged and stirring was continued for 15 min at 25° C. After separation, organic phase was transferred again to vessel A.

Acetonitrile was azeo-dried at 68° C. (700 mmbar ca) under stirring, replacing the solvent distilled off until KF of the reaction mixture was ≦1%.

3.5 vols Et₃N were charged, (more triethylamine can be added to obtain a clear solution) at 70° C., stirring 30 min after dissolution.

Duolite A7 (25% w/w) and Et₃N.HCl (20% w/w) were added.

The slurry was stirred at 70° C. for about 40 hours, checking the reaction by HPLC.

The slurry was cooled to RT, solids were filtered and washed with CH₃CN (2 vols).

Mother liquors were combined, and distilled ⅓ of the total volume at 50° C. and reduced pressure.

After cooling down to 25° C. and adding water (11.5 vols), while stirring for about 15 min, 6M acq. HCl was added till pH of a dilute sample (1:10 with H₂O) of about 1.5.

The mixture was stirred very slowly for about 180 min at 25° C.

The solid was isolated by filtration, washed with water (27 vols ca) till pH of mother liquor ≧6.0, followed by acetonitrile (10 vols) and heptane (3.5 vols).

The solid was dried at 50° C. (0-10 mmbar) for 15 hrs ca; blended solid and dried again at 60° C. (0-10 mmbar) for 15 hrs to obtain a yellow solid with a yield of 60%.

### b. Preparation of Compound of formula (I) (2Z)[2-({4-[(4-acetylpiperazin-1-yl)methyl]benzyl}oxy)pyrimidin-4-yl](1,3-benzothiazol-2-yl)acetonitrile

3.14 kg of 2-({4-[(4-acetylpiperazin-1-yl)methyl]benzyl}oxy) were dissolved in 9.3 liters of dry NMP at 25° C. in a first vessel under N₂.

Separately, 22 liters of dry NMP were charged under N₂ in a second vessel, followed by portion-wise addition of 3.0 kg of the product obtained in step a, stirring the mixture at 45° C. to obtain a solution.

In a third vessel, 1.05 kg of 60% NaH are added 9.0 liters of dry NMP under N₂ at 20° C.

The suspension contained in the third vessel was added the solution of the alcohol (first vessel) in about 30 minutes at 20° C. under N₂ with stirring. The slurry was continued for about 30 minutes at 25° C. and at 45° C. for about 5 minutes.

Last, the solution of product from step a. (second vessel) was loaded under N₂ in about 90 min at 45° C. under stirring.

Reaction mixture was stirred for about 3 hours, and reaction progression was monitored by HPLC after 3 hrs. Reaction mixture was cooled down to −10° C. 38 liters H₂O were added portion-wise maintaining temperature below 20° C.

16 liters heptane were charged while stirring at 20° C. for 15 minutes. Stirring was stopped and phases were let to separate. Organic phase was removed.

1.0-1.5 liters AcOH glac. were added till pH=6.9-7.0, maintaining temperature below 15° C. with stirring and starting precipitation.

25 liters of H₂O were loaded and stirred for about 10 minutes, maintaining temperature below 15° C.

AcOH glac was added till pH=4.5-5.5 and stirring was continued for about 15 hrs at 22° C. very slowly for precipitation formation.

The precipitate was collected by filtration, washed with H₂O (3×18 liters) then with heptane (3×5 liters).

The solid material was dried at 45° C. (0-30 mmbar) for about 15 hours to obtain a yellow solid with a yield equal to 80%.

### Example 2

### Preparation of a salt of Compound of formula (I) (2Z)[2-({4-[(4-acetylpiperazin-1-yl)methyl]benzyl}oxy)pyrimidin-4-yl](1,3-bensothiazol-2yl)acetonitrile, dimethanesulphonate

The product of step b. was dissolved in 10 volumes of AcOH at 23° C., then a solution of 0.3 volumes of methanesulfonic acid and 0.3 volumes of AcOH was added in 15 minutes while stirring at 23° C.

8 volumes of acetone were charged in about 35 min under slow stirring at 23° C. to observe formation of solid material. Slow stirring was continued at 20° C. for about 180 minutes.

8 volumes of acetone were loaded and stirred slowly at 20° C. for about 15 hours.

The solid was filtered and washed with 3.4 volumes of acetone. Dry on filter for 10 min ca under N₂

Charge the solid back to vessel A. Charge 10 volumes of acetone to vessel A and stir at 25° C. for 60 min ca

Filter the solid and wash the cake with 3.4 volumes of acetone. Drying on filter was done under N₂.

The solid was introduced in a vessel and 10 volumes of acetone were loaded thereto and stirring was done at 25° C. for about 60 minutes.

The solid was filtered and washed with 3.4 volumes of acetone. Drying was directly done on the filter for about 20 minutes under N₂.

The solid was stirred with 20 volumes of heptane at 45° C. for about 180 minutes.

The solid was filtered, washed with 10 volumes of heptane and dried on the filter for about 20 minutes under N₂.

The solid material was dried at 55° C. (0-30 mmbar) for about 24 hours to obtain a yellow-orange solid with a yield equal to 75%.

### Example 3

### Preparation of Intermediate (Va where n=1 and R=acetyl) 4-(4-acetyl-piperazin-1-yl-methylphenyl)methanol

5.5 volumes of Toluene were loaded in a glass lined vessel under N₂ and cooled down to −20° C.±2° C.

In a separate vessel, 1.5 kg of methyl-4-bromo-methylbenzoate were charged portion wise while stirring under N₂ at room temperature to obtain a solution (solution B).

19.8 liters (3.02 eq.) of a 1 M solution DIBAL-H/Toluene under N₂ were added, cooling down the solution to −20° C.±2° C. and stirring.

The solution B was loaded under N₂ portion wise while maintaining the temperature in the range 0-15° C. (<35° C.) by addition over about 1 hour.

Reaction was monitored by HPLC when addition was completed.

The mixture was cooled down to −20° C.±2° C. under stirring.

8.8 volumes (2.02 eq.) of 1M acq. HCl (cooled to 5° C.±2° C.) were added drop wise under very slow stirring and maintaining the temperature below 30° C. (<35° C.).

Stirring was stopped and phases separated at 8° C.±2° C.

Water phase was removed.

5 volumes of H₂O were then charged, maintaining the temperature at 10° C.±2° C., very slowly stirring was done for a further 10 minutes.

Stirring was stopped and phases separated at 10° C.±2° C., then removed.

Washing with water and phase separation were repeated.

Toluene was removed by distillation under reduced pressure maintaining solution temperature at 35° C. (<40° C.) to obtain a white solid with yield equal to 90%.

The above solid is dissolved in 7 volumes of dichloromethane in a vessel under N₂ stirring at 25° C. for about 15 minutes. In a separate vessel, 1.05 kg of N-acetyl-piperazine were dissolved in 3 volumes of dichloromethane stirring at 25° C.

Sodium bicarbonate was charged portion wise to the dichloromethane solution under stirring at 23° C.±2° C. in about 10 minutes.

N-acetyl-piperazine solution was loaded to dichlorometane-bicarbonate mixture under stirring at 30° C.±2° C. The mixture was stirred at that temperature for 15 hours, monitoring the reaction by HPLC.

The reaction mixture was cooled down at 23° C.±2° C.

2 volumes of water were added under stirring at 25° C. for about 15 minutes. Stirring was stopped and the phases were separated. Organic phases were separated.

Organic phases were washed with water (2×2 volumes) under stirring for 15 minutes at 25° C. Water phases were collected and washed (2×3 volumes) with dichloromethane under stirring for 15 minutes at 25° C. Organic phases were removed, collected and dried over anhydrous sodium sulfate. The solid cake was washed with 2 volumes of dichloromethane

Dichloromethane solution was concentrated (about 15 volumes at 40° C. under vacuum), subsequently 6 volumes of ethyl acetate were added.

6 volumes of solvent were removed at 65° C.

The solution was cooled down to 53° C. in about 1 hour under slow stirring, then to 5° C.±2° C. in about 2.5 hours under slow stirring to obtain crystallization of the material.

The mixture was filtered at 5° C. and the solid cake washed with 1 volume of ethyl acetate (cooled at 5° C.).

A second crop of material could be obtained from mother liquors by concentration and cooling.

The solid was dried in vacuo in oven (30° C.±2° C.) for about 15 hours.

Average yield 70% (wt) starting from methyl-4-bromo-methylbenzoate, average purity for this step (>97%) on 13 batches.

1-11. (canceled)

12. A process for the preparation of compounds of formula (I) 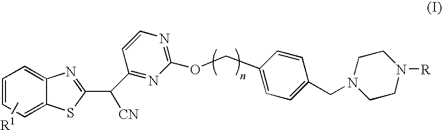
wherein:
R is selected from the group consisting of hydrogen, C₁-C₆ alkyl, C₁-C₆ alkyl aryl, aryl-C₁-C₆ alkyl, heteroaryl, C₁-C₆ alkyl heteroaryl, heteroaryl-C₁-C₆ alkyl,C₂-C₆ alkenyl, C₂-C₆ alkenyl aryl, aryl-C₂-C₆ alkenyl, C₂-C₆ alkenyl heteroaryl, heteroaryl-C₂-C₆ alkenyl, C₂-C₆ alkynyl, C₂-C₆ alkynyl aryl, aryl-C₂-C₆ alkynyl, C₂-C₆ alkynyl heteroaryl, heteroaryl C₂-C₆ alkynyl,C₃-C₈ cycloalkyl, heterocycloalkyl, C₁-C₆ alkyl cycloalkyl, cycloalkyl-C₁-C₆ alkyl,C₁ -C₆ alkyl heterocycloalkyl, heterocycloalkyl-C₁-C₆ alkyl, C₁-C₆ alkyl carboxy, carboxyC₁-C₆ alkyl, acyl, C₁-C₆ alkyl acyl, oxyacyl, C₁-C₆alkyloxyacyl, alkoxy, alkoxycarbonyl, alkylcarboxycarbonyl, aminocarbonyl, C₁-C₆ alkyl aminocarbonyl, aminoacyl, ureido, oxysulfonyl, C₁-C₆alkyl oxysulfonyl, C₁-C₆alkyl sulfonyl, C₁-C₆ alkyl sulfonyl, —C₆ alkyl sulfanyl, aminosulfonyl, and C₁-C₆ alkyl-aminosulfonyl;
R¹ is selected from the group consisting of hydrogen, halogen, cyano, nitro, amino, C₁-C₆ alkyl, heteroaryl, C₂-C₆ alkenyl, C₂-C₆ alkenyl aryl, C₂-C₆ alkenyl heteroaryl, C₂-C₆ alkynyl, C₁-C₆ alkyl aryl, aryl or heteroaryl, C₁-C₆ alkyl heteroaryl, —C(O)—OR², —C(O)—R², —NR²R^{2′}, and —S(O₂)—R², with
R² and R²′ being independently selected from the group consisting of hydrogen, C₁-C₆ alkyl, C₂-C₆ alkenyl, C₂-C₆ alkynyl, aryl, heteroaryl, C₁-C₆-alkyl aryl,aryl-C₁-C₆ alkyl C₁-C₆-alkyl heteroaryl, and heteroaryl-C₁-C₆-alkyl;
n is an integer from 0 to 3;
comprising:
a) reacting a benzothiazol-2-ylacetonitrile bearing group R₁ (III) with an activated pyrimidine (VI) to obtain a halogenopyrimidino-benzothiazole derivative (IV) in a reaction medium; 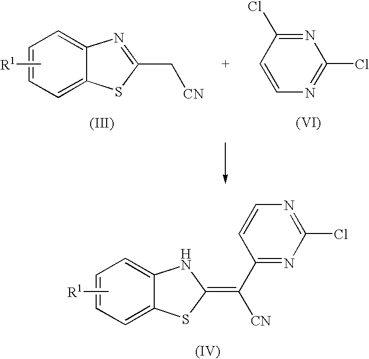
b) treating the derivative (IV) obtained in step a) in said reaction medium with a weak base anion exchange resin;
c) removing said resin from said reaction medium;
d) isolating compound (IV); and
e) reacting compound (IV) with a substituted piperazine-benzyl-alkyloxy (V) in a reaction medium to give a compound of formula (I);

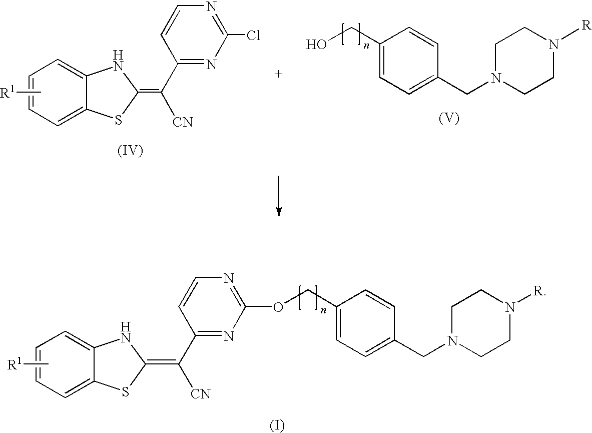

13. The process according to claim 12, further comprising salifying compound (I) to form a pharmaceutically acceptable salt.

14. The process according to claim 12, wherein, in formula (I), R is acyl and n is 1.

15. The process according to claim 14, wherein R is acetyl.

16. The process according to claim 12, wherein, in formula (I), R is acyl, n is 1 and R₁ is hydrogen.

17. The process according to claim 16, wherein [2-({4-[(4-acetylpiperazin-1-yl)methyl]benzyl}oxy)-pyrimidin-4-yl](1,3-benzothiazol-2-yl)acetonitrile is the compound of formula (I).

18. The process according to claim 13, wherein the pharmaceutically acceptable salt is selected from the group consisting of dimethanesulfonate, mesylate and trifluoracetate.

19. The process according to claim 12, wherein the activated pyrimidine (VI) is a dihalogenopyrimidine.

20. The process according to claim 12, wherein the reaction medium in step a) is selected from the group consisting of acetonitrile and N-methylpyrrolidone.

21. The process according to claim 12, wherein the reaction medium in step e) is N-methylpyrrolidone.

22. A process for the preparation of a substituted piperazine-benzyl-alkyloxy of formula (V) 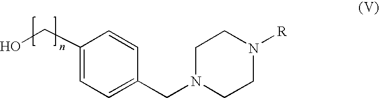
wherein R is an acyl group, said process comprising:
a) treating a bromide-alkyl-phenyl-4-ester wherein the ester group is selected from COOMe or COOEt with DIBAL to obtain (4-bromomethyl-phenyl)-methanol; and
b) reacting the (4-bromomethyl-phenyl)-methanol obtained in step a) with 1-piperazin-1-yl-acyl.

23. A process for the preparation of compounds of formula (I), 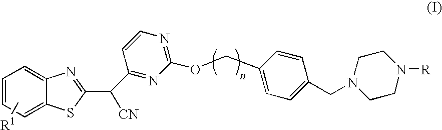
a) wherein R is acyl, R₁ is as defined in claim 1, comprising the following steps:
b) treating a bromide-alkyl-phenyl-4-ester wherein the ester group is selected from COOMe or COOEt with DIBAL to obtain (4-bromomethyl-phenyl)-methanol;
c) reacting the (4-bromomethyl-phenyl)-methanol obtained in step a) with 1-piperazin-1-yl-acyl to give 1-[4-(4-Hydroxymethyl-benzyl)-piperazin-1-yl]-acyl (V); and
d) reacting said 1-[4-(4-Hydroxymethyl-benzyl)-piperazin-1-yl]-acyl (V) obtained in step b) with a halogeno-pyrimidino-benzothiazole derivative (IV) to give a compound of formula (I).

24. The process according to claim 23, further comprising removing group R.

25. The process according to claim 23, further comprising salifying compound (I).

26. The process according to claim 24, further comprising salifying the compound after the removal of group R.
